(19) United States
(12) Patent Application Publication
(10) Pub. No.: US 2024/0381992 A1
(43) Pub. Date: Nov. 21, 2024

(54) Dental Appliance Camera Device

(71) Applicant: Ervin Burris, Chapin, SC (US)

(72) Inventor: Ervin Burris, Chapin, SC (US)

(21) Appl. No.: 18/664,437

(22) Filed: May 15, 2024

Related U.S. Application Data
(60) Provisional application No. 63/502,245, filed on May 15, 2023.

(51) Int. Cl. A46B 15/00; A61B 1/00; A61B 1/06; A61B 1/24; H04N 7/18

(52) U.S. Cl. CPC A46B 15/0022; A46B 15/0036; A61B 1/00016; A61B 1/00034; A61B 1/0655; A61B 1/24; H04N 7/183; A46B 2200/1066

(57) ABSTRACT

A dental appliance camera device is provided. The device is comprised of a multifunctional dental device comprising a body and an appliance head, wherein the appliance head may be a toothbrush or water sprayer head. The body attaches to the appliance head and may feature articulation points allowing movement in multiple directions. The body is also comprised of a camera and a wireless transmitter for streaming photos and video to a mobile application. As a result, the device allows users and/or dental professionals to view and record the conditions inside the mouth during teeth brushing and/or flossing, facilitating better dental health management.

### CROSS-REFERENCE TO RELATED APPLICATION

The present application claims priority to, and the benefit of, U.S. Provisional Application No. 63/502,245, which was filed on May 15, 2023, and is incorporated herein by reference in its entirety.

### FIELD OF THE INVENTION

The present invention relates generally to the field of dental appliances. More specifically, the present invention relates to a dental appliance camera device that can be applied to or is comprised of a toothbrush head and/or water sprayer head, wherein the device provides photo and/or video to a mobile application while using the toothbrush or water sprayer. Accordingly, the present disclosure makes specific reference thereto. Nonetheless, it is to be appreciated that aspects of the present invention are also equally applicable to other like applications, devices, and methods of manufacture.

### BACKGROUND

Standard toothbrushes and other dental appliances of the lack do not allow users to visually inspect the interior of their mouth during use of the device. This capability could help identify unclean areas and spots that might require additional brushing or dental care. Without such technology, individuals may remain unaware of potential oral health issues, missing early signs of dental problems that could be addressed more efficiently with better visibility.

Therefore, there exists a long-felt need in the art for an improved dental device. There also exists a long-felt need in the art for a dental appliance camera device. More specifically, there exists a long-felt need in the art for a dental appliance camera device that provides a user with a recorded video of the inside of the mouth while using the device.

The subject matter disclosed and claimed herein, in one embodiment thereof, comprises a dental appliance camera device. The device is comprised of a multifunctional dental device comprising a body and an appliance head, wherein the appliance head may be a toothbrush or water sprayer head. The body attaches to the appliance head and may feature articulation points allowing movement in multiple directions. The body is also comprised of a camera and a wireless transmitter for streaming photos and video to a mobile application. As a result, the device allows users and/or dental professionals to view and record the conditions inside the mouth during teeth brushing and/or flossing, facilitating better dental health management.

In this manner, the dental appliance camera device of the present invention accomplishes all the forgoing objectives and provides an improved dental device. More specifically, the device does so by providing a user with a recorded video of the inside of the mouth while using the device. This video can then be viewed by the user and/or a third party, such as but not limited to a dental professional.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a dental appliance camera device comprised of a body. The body may be made of any material and may be any shape. The body preferably attaches to an appliance head such as but not limited to a toothbrush head, a water sprayer head, etc. The body preferably snap fits onto/around the head in a removable fashion.

In one embodiment, the head may have at least one articulation point that allows the head to move at least one degree in at least one direction (such as but not limited to the vertical and/or horizontal direction once attached) to an appliance body. The body may include but is not limited to any style of toothbrush body, water sprayer body, etc. with any features, functions, and structures associated with said bodies and devices that are known in the art.

The body is comprised of at least one camera that can stream wirelessly photos and/or video to a mobile application via at least one transmitter in wireless communication with the smart device the mobile application is installed on. This allows photos/videos of the interior of a user's mouth to be streamed, viewed, and/or recorded by the mobile application while using the device.

The quality of the video from the camera may be enhanced via at least one light positioned anywhere on the body. In one embodiment, the light has at least one brightness level that can be controlled/selected via at least one button on the body and/or controlled by the mobile application.

As a result, the device can be used while brushing and/or flossing in order to view the inside of the user's mouth, teeth, gums, tongue, etc. Then, the video from said activity can be viewed from the mobile application viewed from the mobile application by the user and/or a dental professional for record keeping and/or further evaluation.

Accordingly, the dental appliance camera device of the present invention is particularly advantageous as it provides an improved dental device. More specifically, the device does so by providing a user with a recorded video of the inside of the mouth while using the device. This video can then be viewed by the user and/or a third party, such as but not limited to a dental professional. In this manner, the dental appliance camera device overcomes the limitations of existing dental appliances known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of a body of one potential embodiment of a dental appliance camera device of the present invention in accordance with the disclosed architecture;

FIG. 2 illustrates a front view and a side view of a body of one potential embodiment of a dental appliance camera device of the present invention while attached to an appliance head in accordance with the disclosed architecture;

FIG. 3 illustrates a front view of a body of one potential embodiment of a dental appliance camera device of the present invention while attached to an appliance head and wherein the appliance head is attached to an appliance body in accordance with the disclosed architecture; and

FIG. 4 illustrates a graphical view of features of a mobile application of one potential embodiment of a dental appliance camera device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved dental device. There also exists a long-felt need in the art for a dental appliance camera device. More specifically, there exists a long-felt need in the art for a dental appliance camera device that provides a user with a recorded video of the inside of the mouth while using the device.

The present invention, in one exemplary embodiment, is comprised of a dental appliance camera device. The device is comprised of a body that preferably attaches to an appliance head such as but not limited to a toothbrush head, a water sprayer head, etc. The body preferably snap fits onto/around the head in a removable fashion.

In one embodiment, the head may have at least one articulation point that allows the head to move at least one degree in at least one direction (such as but not limited to the vertical and/or horizontal direction) once attached to an appliance body. The body may include but is not limited to any style of toothbrush body, water sprayer body, etc. with any features, functions, and structures associated with said bodies and devices that are known in the art.

The body is comprised of at least one camera that can stream wirelessly photos and/or video to a mobile application via at least one transmitter in wireless communication with the smart device the mobile application is installed on. This allows photos/videos of the interior of a user's mouth to be streamed, viewed, and/or recorded by the mobile application while using the device.

The quality of the video from the camera may be enhanced via at least one light positioned anywhere on the body. In one embodiment, the light has at least one brightness level that can be controlled/selected via at least one button on the body and/or controlled by the mobile application.

As a result, the device can be used while brushing and/or flossing in order to view the inside of the user's mouth, teeth, gums, tongue, etc. Then, the video from said activity can be viewed from the mobile application viewed from the mobile application 130 by the user and/or a dental professional for record keeping and/or further evaluation.

Accordingly, the dental appliance camera device of the present invention is particularly advantageous as it provides an improved dental device. More specifically, the device does so by providing a user with a recorded video of the inside of the mouth while using the device. This video can then be viewed by the user and/or a third party, such as but not limited to a dental professional. In this manner, the dental appliance camera device overcomes the limitations of existing dental appliances known in the art.

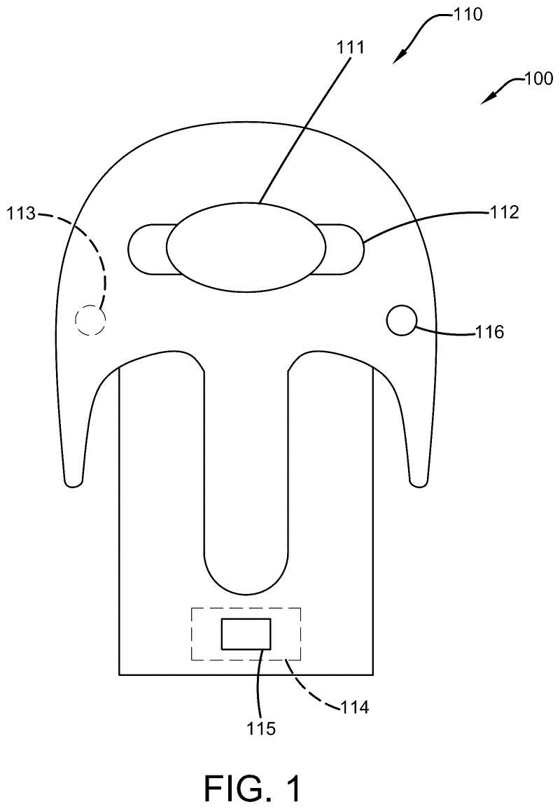

Referring initially to the drawings, FIG. 1 illustrates a front view of a body 110 of one potential embodiment of a dental appliance camera device 100 of the present invention in accordance with the disclosed architecture. The device 100 is comprised of a body 110. The body 110 may be made of any material and may be any shape. In one embodiment, the body 110 is made from an anti-bacterial material.

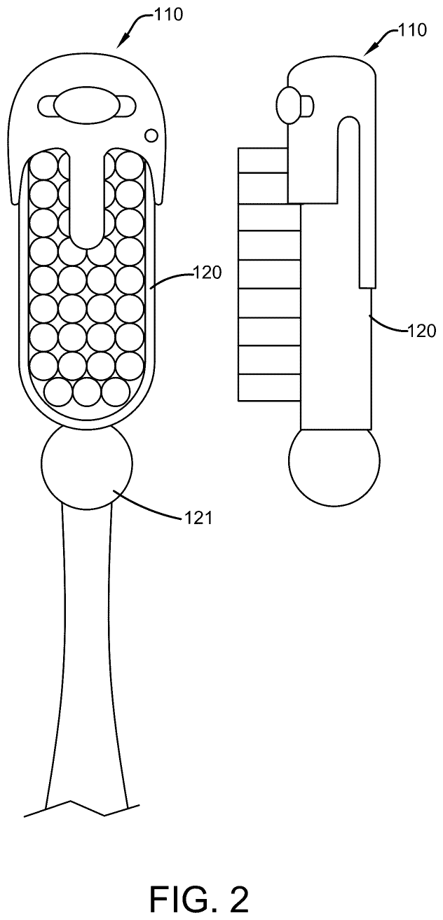

The body 110 preferably attaches to an appliance head 120. The appliance head 120 may be but is not limited to a toothbrush head, a water sprayer head, etc. The body 110 preferably snap fits onto/around the head 120 in a removable fashion to allow for the body 110 to be used on multiple heads 120 of different dental appliances, as seen in FIG. 2. In a different embodiment, the body 110 is fixedly attached to the head 120. The head 120 may be specifically shaped and configured in one embodiment to receive the body 110.

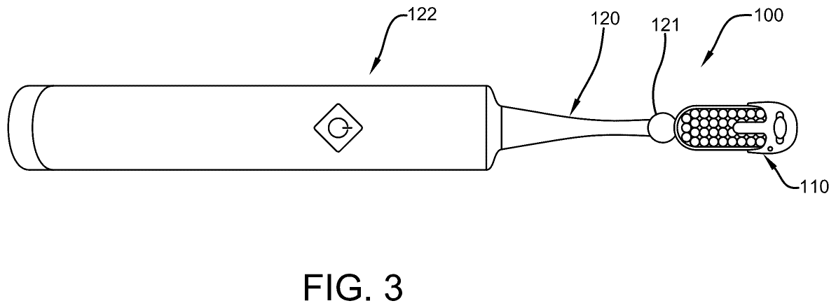

In one embodiment, the head 120 may have at least one articulation point 121 that allows the head 120 to move at least one degree in at least one direction (such as but not limited to the vertical and/or horizontal direction) once attached to an appliance body 122, as seen in FIG. 3. The body 122 may include but is not limited to any style of toothbrush body, water sprayer body, etc. with any features, functions, and structures associated with said bodies and devices that are known in the art. More specifically, an embodiment wherein the body 122 is a water sprayer body preferably has a reservoir for storing a liquid and then spraying said liquid into the mouth of a user to clean the teeth/mouth area. The head 120 may fixedly or removably attach to the body 122 in different embodiments.

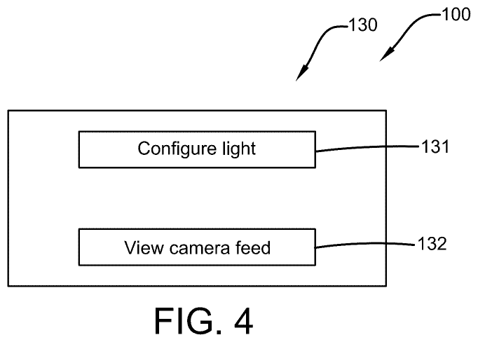

The body 110 is comprised of at least one camera 111. The camera 111 may be any type and configuration of camera that can stream wirelessly photos and/or video to a mobile application 130 (wherein the functions of the application 130 can be seen in FIG. 4) via at least one transmitter 113 such as but not limited to a Bluetooth transmitter 113. The transmitter 113 is in wireless communication with the smart device the application 130 is installed on. This allows photos/videos of the interior of a user's mouth to be streamed, viewed 132, and/or recorded by the mobile application 130 while using the device 100.

The quality of the video from the camera 111 may be enhanced via at least one light 112 positioned anywhere on the body 110. The light 112 may be any type of light and may illuminate any color. In one embodiment, the light has at least one brightness level that can be controlled/selected via at least one button 116 on the body 110 and/or controlled 131 by the mobile application 130.

The camera 111 and light 112 may be powered via at least one battery 114. The battery 114 may be removable or non-removable. The battery 114 may also be a rechargeable or a non-rechargeable battery. In a non-removable and rechargeable embodiment, the battery 114 may be recharged via at least one waterproof charging port 115. The charging port 115 may be any type such as but not limited to a USB port.

As a result, the device 100 can be used while brushing and/or flossing in order to view the inside of the user's mouth, teeth, gums, tongue, etc. Then, the video from said activity can be viewed from the mobile application viewed 132 from the mobile application 130 by the user and/or a dental professional.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “dental appliance camera device” and “device” are interchangeable and refer to the dental appliance camera device 100 of the present invention.

Notwithstanding the forgoing, the dental appliance camera device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the dental appliance camera device 100 as shown in the FIGS. are for illustrative purposes only, and that many other sizes and shapes of the dental appliance camera device 100 are well within the scope of the present disclosure. Although the dimensions of the dental appliance camera device 100 are important design parameters for user convenience, the dental appliance camera device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A dental appliance camera device comprising:
a body comprised of a camera, a light, a transmitter, and a battery; and
a mobile application.

2. The dental appliance camera device of claim 1, wherein the body is comprised of a charging port for the battery.

3. The dental appliance camera device of claim 1, wherein a brightness level of the light can be configured by the mobile application.

4. The dental appliance camera device of claim 1, wherein a photo or a video from the camera is streamed to the mobile application via the transmitter.

5. The dental appliance camera device of claim 1, wherein the transmitter is comprised of a Bluetooth transmitter.

6. A dental appliance camera device comprising:
a body comprised of a camera, a light, a transmitter, and a battery;
an appliance head; and
a mobile application.

7. The dental appliance camera device of claim 6, wherein the appliance head is comprised of a toothbrush head.

8. The dental appliance camera device of claim 6, wherein the appliance head is comprised of a water sprayer head.

9. A dental appliance camera device comprising:
a body comprised of a camera, a light, a transmitter, and a battery;
an appliance head;
an appliance body; and
a mobile application.

10. The dental appliance camera device of claim 9, wherein the appliance head is comprised of a toothbrush head.

11. The dental appliance camera device of claim 9, wherein the appliance head is comprised of a water sprayer head.

12. The dental appliance camera device of claim 9, wherein the appliance body is comprised of a toothbrush body.

13. The dental appliance camera device of claim 9, wherein the appliance head is comprised of a water sprayer body.

14. The dental appliance camera device of claim 9, wherein the appliance head attaches to the appliance body via a joint.

15. The dental appliance camera device of claim 14, wherein the appliance head articulates a degree in a horizontal direction relative to the joint.

16. The dental appliance camera device of claim 14, wherein the appliance head articulates a degree in a vertical direction relative to the joint.

17. The dental appliance camera device of claim 9, wherein the body is comprised of a button.

18. The dental appliance camera device of claim 17, wherein the buttons control a brightness level of the light.

19. The dental appliance camera device of claim 9, wherein the body removably attaches to the appliance head.

20. The dental appliance camera device of claim 9, wherein the body is comprised of a waterproof charging port.
